(19) United States
(12) Patent Application Publication
(10) Pub. No.: US 2017/0319747 A1
(43) Pub. Date: Nov. 9, 2017

(54) THREE-DIMENSIONAL CULTURE METHOD USING BIODEGRADABLE POLYMER AND CULTURE SUBSTRATE ENABLING CELL TRANSPLANTATION

(71) Applicants: KYOTO UNIVERSITY, Kyoto-shi, Kyoto (JP); GUNZE LIMITED, Ayabe-shi, Kyoto (JP)

(72) Inventors: Kenichiro KAMEI, Kyoto-shi, Kyoto (JP); Li LIU, Kyoto-shi, Kyoto (JP); Norio NAKATSUJI, Kyoto-shi, Kyoto (JP); Yong CHEN, Kyoto-shi, Kyoto (JP); Hideki SATO, Ayabe-shi, Kyoto (JP); Masakazu SUZUKI, Ayabe-shi, Kyoto (JP)

(73) Assignees: KYOTO UNIVERSITY, Kyoto-shi, Kyoto (JP); GUNZE LIMITED, Ayabe-shi, Kyoto (JP)

(21) Appl. No.: 15/522,189

(22) PCT Filed: Oct. 30, 2015
(86) PCT No.: PCT/JP2015/080641

§ 371 Date: Apr. 26, 2017

(30) Foreign Application Priority Data
Oct. 31, 2014 (JP) 2014-223702

(51) Int. Cl. A61L 27/38; A61L 27/58; C12N 5/0735

(52) U.S. Cl. CPC A61L 27/3834; C12N 5/0606; A61L 27/58; A61L 27/3895; C12N 2513/00; A61L 2300/64; A61L 2400/18; C12N 2533/40; C12N 2533/54; C12N 2537/10

(57) ABSTRACT

The present invention provides a cell culture substrate containing a nanofiber composed of a biodegradable polymer on a support composed of a biodegradable polymer. It also provides a method of culturing cells, which includes seeding cells on the substrate, and stationary culture of the cells. Furthermore, the present invention provides an agent for cell transplantation therapy, which contains the substrate and cells cultured on the substrate.

### TECHNICAL FIELD

The present invention relates to three-dimensional culture of a cell, for example, a stem cell including pluripotent stem cells such as embryonic stem cells (ES cells), induced pluripotent stem cells (iPS cells) and the like, particularly human pluripotent stem cells, as well as a culture substrate permitting direct transplantation of cells to the living body without detachment, a method of culturing cells by using the culture substrate, a safe agent for a cell transplantation therapy, which is obtained by the method, and the like. More particularly, the present invention relates to a substrate for cell culture comprising a biodegradable polymer support coated with a nanofiber composed of a biodegradable polymer, a method of maintenance and amplification of cells, comprising dispersing the cells into single cells by using the culture substrate, and without performing an enzyme treatment during passage, an agent for cell transplantation therapy comprising the culture substrate and cells cultured on the substrate, and the like.

### BACKGROUND ART

Human pluripotent stem cells are capable of unlimited proliferation under appropriate conditions and have the property to differentiate into any cell of biological tissues (multipotency). Therefore, application to various fields such as cell transplantation therapy, drug discovery screening, regenerative medicine and the like is expected. In conventional culture methods for human pluripotent stem cells, feeder cells and various macromolecules have been used as cell culture substrates. However, since these methods require complicated preparative operations and fail to afford stable quality, stable culture and supply of human pluripotent stem cells has been difficult to achieve. In particular, a more stable and inexpensive method is necessary for developing a high-quality, large-scale, fully-automated culture method of human pluripotent stem cells. However, such method has not yet been established.

Conventionally-performed two-dimensional culture using culture dishes is not suitable for the development of a high-quality, large-scale, fully-automated culture method of human pluripotent stem cells for the reasons that culture dishes in a unit of 100 are necessary, a passage operation of individual culture dishes is necessary, and the like. To enable large-scale culture of pluripotent stem cells in a limited space, therefore, three-dimensional culture is essential. While suspension culture and culturing methods using microbeads and the like have been developed heretofore (non-patent documents 1, 2), they have not been put to practical use due to the problems of aggregation of cell mass, shear stress on the cell surface due to agitation and the like.

In recent years, a novel culture method of human pluripotent stem cells, which does not use feeder cells, has been actively developed. At present, Matrigel, recombinant proteins (non-patent document 3) and the like have been widely used as cell culture substrates. However, these materials are costly and lack stability due to large differences in quality between lots, and the like.

Human pluripotent stem cells cultured under such conditions are unstable, as a result of which abnormalities such as abnormal cell proliferation rate, degeneration to a highly non-uniform cell population, loss of differentiation potency, karyotype mutation and the like occur.

As an alternative, the development of a cell culture substrate using a macromolecule such as a polymer and the like has also been reported (non-patent documents 4, 5) and commercialized. Although stable products can be obtained, they are very expensive and are sometimes unsuitable depending on the cell line. Accordingly, a stable and inexpensive cell culture substrate has not been produced yet.

The cell culture substrate is required to supply necessary oxygen and nutrients to the target cell population and maintain a stable shape. In recent years, nanofibers have attracted attention. Nanofibers are ultrafine fibers with fiber diameters on the order of nanometers. Structures composed of nanofibers have a size approximate to that of a extracellular matrix, and are advantageous in that the cell adhesiveness is improved by an increase in the specific surface area, three-dimensional culture becomes possible and the like. Therefore, a synthetic polymer (non-patent document 6) and a nanofiber composed of a mixture of a synthetic polymer and a biological macromolecule such as collagen, gelatin and the like (non-patent documents 6, 7) have been produced. However, it has been reported that a culture system without using feeder cells cannot maintain and grow human ES cells (non-patent document 7).

Conventionally, for the passage of human pluripotent stem cells, a method using enzymes such as collagenase, dispase, trypsin or the like, or a mechanical passage method by a cell strainer, pipetting and the like has been performed. In a method using an enzyme, cells are damaged by an enzymatic reaction, and the enzymatic reaction on the cells is non-uniform. Moreover, when cells are dispersed into single cells, a problem of cell death occurs. On the other hand, the mechanical passage method causes a very large damage on the cells and has many problems.

The present inventors took note of the use of a highly biocompatible and inexpensive biomaterial as a substrate for culturing human pluripotent stem cells, and made a nanofiber of a biomaterial by using the electrospinning method (patent document 1). Human pluripotent stem cells cultured on the nanofiber substrate showed superior proliferation equivalent to that of culture on Matrigel. Furthermore, it has been clarified that, when passage culturing is performed using the nanofiber substrate, the cells can be dispersed into single cells only by a slight pipetting operation without performing an enzyme treatment, and the cell death seen in the conventional method can be remarkably suppressed.

### DOCUMENT LIST

### Patent Document

patent document 1: JP-A-2013-247943

### Non-Patent Documents

non-patent document 1: Tissue Eng Part C Methods, 16(4), 573-582 (2010)
non-patent document 2: Curr Protoc Stem Cell Biol Chapter 1, Unit 1C 11 (2010)
non-patent document 3: Nature Biotechnology, 28(6): 581-583 (2010)
non-patent document 4: Nature Biotechnology, 28(6): 606-610 (2010)
non-patent document 5: Nature Biotechnology, 28(6): 611-615 (2010)
non-patent document 6: Advanced Drug Delivery Reviews, 61(12): 1084-1096 (2009)
non-patent document 7: Journal of Cellular and Molecular Medicine, 13(9B): 3475-3484 (2009)

### SUMMARY OF THE INVENTION

### Problems to be Solved by the Invention

The biological macromolecule nanofiber created by the present inventors has been shown to be a culture substrate suitable for culturing human pluripotent stem cells. However, it was not sufficient for three-dimensional mass culture because of the use of a support lacking plasticity such as glass and the like. In addition, when somatic cells induced to differentiate from human pluripotent stem cells are used for cell transplantation therapy, it was necessary to once detach nanofibers carrying the graft cells from the support, even when a biodegradable biological macromolecule such as gelatin is used as the nanofiber.

Therefore, a first object of the present invention is to provide a novel culture substrate suitable for three-dimensional mass culture, which can stably supply a large amount of cells including human pluripotent stem cells.

A second object of the present invention is to provide a culture substrate which can be transplanted directly to the body without detaching the cells, and a safe agent for cell transplantation therapy, comprising the culture substrate and graft cells cultured on the substrate.

### Means of Solving the Problems

To achieve the first object mentioned above, the present inventors have already invented a substrate for culture wherein biological macromolecule nanofibers are applied on a microfiber support such as gauze, sponge and the like composed of a biocompatible material such as cotton and the like (named “fiber-on-fiber”) (PCT/JP2014/064789). Since fiber-on-fiber can change its shape flexibly, it can also be folded and used. In addition, since gauze, sponge and the like are porous as compared to glass or plastic base materials and the like, when the fiber-on-fiber is immersed in a culture medium, the culture medium naturally penetrates and supply of the culture medium to the cells is also improved. In addition, since the fiber-on-fiber is flexible in shape, selection of a container is not necessary. As long as nutrients reach the cells, the cells can be cultured in any container, and desired cells such as stem cells including pluripotent stem cells can be easily cultured in large quantities.

However, in fiber-on-fiber composed of gelatin nanofibers formed on a cotton gauze support, the cell proliferation per unit area of human ES cells was somewhat inferior to that of gelatin nanofiber formed on Matrigel or a glass support. In addition, the fiber-on-fiber could not be used as it was for cell transplantation.

Thus, the present inventors have produced fiber-on-fiber substrate wherein a biodegradable polymer such as polyglycolic acid (PGA) and the like is used as a microfiber support instead of materials such as cotton and the like and, on the support, a nanofiber also composed of a biodegradable polymer such as gelatin, PGA and the like is applied, and cultured human pluripotent stem cells. As a result, the biodegradable fiber-on-fiber unexpectedly increased remarkably the growth rate of human pluripotent stem cells per unit area as compared to a fiber-on-fiber containing conventional non-biodegradable microfiber. In addition, it was confirmed that human pluripotent stem cells, which were three-dimensionally cultured by folding the biodegradable fiber-on-fiber, maintained pluripotency and normal karyotype even after long-term passage culture.

Furthermore, when human pluripotent stem cells cultured on the biodegradable fiber-on-fiber were transplanted into immunodeficient mice, it was confirmed that a teratoma was formed about 2 months later, and the cell line of all three germ layers was contained therein. In addition, an inflammatory reaction did not occur at all in the transplanted site. Furthermore, necrosis of the grafted cells was not found and fiber-on-fiber completely disappeared in the teratomas. Therefore, it was confirmed that the biodegradable fiber-on-fiber of the present invention is highly safe, does not adversely affect the differentiation of human pluripotent stem cells and can be used for transplantation.

Based on these findings, the present inventors conducted further studies and completed the present invention.

That is, the present invention is as follows.
[1] A cell culture substrate comprising a nanofiber composed of a biodegradable polymer on a support composed of a biodegradable polymer.
[2] The substrate of the above-mentioned [1], wherein the nanofiber is crosslinked.
[3] The substrate of the above-mentioned [1] or [2], wherein the biodegradable polymer constituting the support is a synthetic polymer.
[4] The substrate of the above-mentioned [3], wherein the synthetic polymer is selected from the group consisting of polyester, polycarbonate and a copolymer thereof, polyanhydride and a copolymer thereof, polyorthoester, and polyphosphazen.
[5] The substrate of the above-mentioned [3], wherein the synthetic polymer is polyglycolic acid (PGA).
[6] The substrate of any of the above-mentioned [1]-[5], wherein the support is a non-woven fabric.
[7] The substrate of any of the above-mentioned [1]-[6], wherein the biodegradable polymer constituting the nanofiber is gelatin or a synthetic polymer.
[8] The substrate of the above-mentioned [7], wherein the synthetic polymer is PGA.
[9] The substrate of any of the above-mentioned [1]-[8], wherein the nanofiber is obtained by an electrospinning method.
[10] The substrate of any of the above-mentioned [1]-[9], wherein the cell is a stem cell.
[11] The substrate of the above-mentioned [10], wherein the stem cell is a pluripotent stem cell.
[12] The substrate of the above-mentioned [11], wherein the pluripotent stem cell is an ES cell or iPS cell.
[13] The substrate of the above-mentioned [11] or [12], wherein the pluripotent stem cell is derived from human.
[14] The substrate of any of the above-mentioned [1]-[13], wherein the culture is maintenance and amplification of the cell.
[15] The substrate of any of the above-mentioned [1]-[13], wherein the culture is differentiation induction of pluripotent stem cells.
[16] A method of culturing cells comprising seeding cells on the substrate of any of the above-mentioned [1]-[9], and subjecting the cells to stationary culture.
[17] The method of the above-mentioned [16], comprising dissociating the cells from the substrate by using a dissociation solution free of an enzyme, seeding the cells on the substrate of any of the above-mentioned [1]-[9], and further subjecting the cells to stationary culture.
[18] The method of the above-mentioned [17], wherein the cells are dispersed into single cells during passage.
[19] The method of any of the above-mentioned [16]-[18], wherein the cells are cultured in a xeno-free medium.
[20] The method of the above-mentioned [19], wherein the medium is a protein-free medium.
[21] The method of any of the above-mentioned [16]-[20], wherein the cell is a stem cell.
[22] The method of the above-mentioned [21], wherein the stem cell is a pluripotent stem cell.
[23] The method of the above-mentioned [22], wherein the pluripotent stem cell is an ES cell or iPS cell.
[24] The method of the above-mentioned [22] or [23], wherein the pluripotent stem cell is derived from human.
[25] The method of any of the above-mentioned [16]-[24], wherein the culture is maintenance and amplification of the cell.
[26] The method of any of the above-mentioned [16]-[24], wherein the culture is differentiation induction of pluripotent stem cells.
[27] An agent for a cell transplantation therapy, comprising the substrate of any of the above-mentioned [1]-[9], and cells cultured on the substrate.
[28] The agent of the above-mentioned [27], wherein the cell was induced to differentiate from a pluripotent stem cell.

### Effect of the Invention

The culture substrate of the present invention has high physical strength and is flexible in shape. Therefore, three-dimensional culture becomes possible, and supply of a large amount of cells is possible while realizing space saving. In addition, since the culture substrate of the present invention has high biocompatibility and is inexpensive, stable supply is facilitated. Furthermore, since the culture substrate of the present invention can easily change its shape, it can be cryopreserved regardless of the container.

In addition, since the culture substrate of the present invention is composed of a biodegradable polymer, cell transplantation is possible as it is.

Such culture substrate capable of mass culture/cell transplantation can greatly contribute to the development of regenerative medicine, tissue engineering and cell transplantation treatment. A larger tissue requires a large amount of cells, and a cell detachment operation not only damages cells and tissues, but also destroys even a produced tissue structure. Therefore, transplantation of the cultured cells as they are is useful for avoid this problem. It is also useful that the substrate is decomposed after a while posttransplantation, since it reduces an influence on the patients.

### BRIEF DESCRIPTION OF THE DRAWINGS

FIG. 1 is shows electron microscope photographs (no crosslinking treatment: middle panel, with crosslinking treatment: right panel) of biodegradable fiber-on-fiber obtained by applying gelatin nanofibers on PGA non-woven fabric, and an electron microscope photograph (left) of PGA non-woven fabric.

FIG. 2 is a photograph of human ES cells (H1) cultured on a biodegradable fiber-on-fiber and stained with alkaline phosphatase as a pluripotent stem cell marker.

FIG. 3 shows comparison of cell proliferation rates when human ES cells (H1) were cultured using various gelatin nanofibers. -♦-: cultured on Matrigel, -▪-cultured on nanofiber formed on glass, -▴-: cultured on nanofiber formed on cotton gauze, -◯-: cultured on nanofiber formed on PGF non-woven fabric

FIG. 4 shows the results of flow cytometry showing expression of undifferentiation markers (SSEA4, TRA-1-60) and differentiation marker (SSEA1) in human ES cells (H1, H9) and human iPS cells (253G1) cultured on fiber-on-fiber.

FIG. 5 shows immunocyte staining photograph (right panel) showing expression of undifferentiation marker (OCT4) and differentiation marker (SSEA1) in human ES cells (H1) cultured on fiber-on-fiber. Left panel shows bright field observation, and the middle panel is a photograph of nuclear staining with DAPI.

FIG. 6 shows that teratoma excised from an immunodeficient mouse transplanted with human ES cells (H1; upper) and human iPS cells (253G1; lower) cultured on biodegradable fiber-on-fiber, including substrate, has the cells of all cell lines of three germ layers (neuroepithelium (ectoderm), cartilage (mesoderm), the intestine-like epithelium (endoderm) from the left).

FIG. 7 is an electron microscope photograph of fiber-on-fiber constituted only of PGA.

FIG. 8 is a photograph of human iPS cells (253G1) cultured on fiber-on-fiber constituted only of PGA, and stained with alkaline phosphatase as a pluripotent stem cell marker.

FIG. 9 shows the results of flow cytometry showing the expression of undifferentiation markers (TRA-1-60: left, SSEA4: right) in human iPS cells (253G1) cultured on fiber-on-fiber constituted only of PGA.

FIG. 10 shows substance diffusion behavior via fiber-on-fiber (FoF) constituted only of PGA.

### DESCRIPTION OF EMBODIMENTS

The present invention provides a cell culture substrate containing a nanofiber composed of a biodegradable polymer on a support composed of a biodegradable polymer (hereinafter sometimes to be abbreviated as the culture substrate of the present invention).

### I. Cell

The cell to which the culture substrate of the present invention is applicable is not particularly limited, and substrate can be used for any cell capable of stationary culture (e.g., differentiated cells of lymphocyte, epithelial cell, endothelial cell, myocytes, fibroblast (skin cell etc.), bristle cell, hepatocyte, bristle cell, intestinal cell, splenocyte, pancreatic cell (exocrine pancreas cell etc.), brain cell, pneumocyte, nephrocyte, adipocyte and the like, undifferentiated tissue progenitor cell, stem cell and the like).

In one preferable embodiment, stem cell can be mentioned. The stem cell is not particularly limited as long as it has a self-replication ability and an ability to differentiate into other kind of cell (other than stem cell). Any of pluripotent stem cells capable of differentiating into all lines of three germ layers, stem cells which cannot generally differentiate beyond the germ layer but have multipotency capable of differentiating into various cytomas, and unipotent stem cells that can differentiate into one limited type of cytoma are applicable.

The pluripotent stem cell is not particularly limited as long as it is an undifferentiated cell having “self-renewal ability” enabling proliferation while maintaining an undifferentiated state and “differentiation pluripotency” enabling differentiation into all lines of three germ layers. For example, it may be ES cell, iPS cell, embryonic germ (EG) cell derived from primordial germ cell, multipotent germline stem (mGS) cell isolated in the GS cell establishing culture process from testis tissue, multipotent adult progenitor cell (MAPC) isolated from bone marrow, pluripotent cell (Muse cell) derived from culture fibroblast and myelogenic stem cell, or the like. ES cell may be a nuclear transfer ES (ntES) cell produced by nuclear reprogramming of somatic cell. Preferred is an ES cell or iPS cell.

Examples of the stem cell having multipotency include, but are not limited to, neural stem cell, hematopoietic stem cell, mesenchymal stem cell, liver stem cell, pancreas stem cell, skin stem cell and the like. Examples of the unipotent stem cell include, but are not limited to, muscle stem cell, germ stem cell, dental pulp stem cell and the like.

When the cell cultured by the method of the present invention is a differentiated cell, tissue progenitor cell, stem cell having multipotency, or unipotent stem cell, these cells can be isolated by a method known per se from a tissue of any mammal in which they are present. The isolated cell can be applied as it is as a primary cultured cell, or applied after maintenance culture by a method known per se. In addition, various cell strains obtained by immortalizing cultured cells thereof can also be used.

On the other hand, when the cell is a pluripotent stem cell, the method of the present invention can be applied to any mammal in which some pluripotent stem cell has been established or can be established, and examples thereof include human, mouse, monkey, swine, rat, dog and the like. Preferred is human or mouse, more preferred is human. While preparation methods of various pluripotent stem cells are specifically explained below, other known methods can also be used without limitation.

ES cell can be established by taking out an inner cell mass from the blastocyst of a fertilized egg of the target animal, and culturing the inner cell mass on a feeder of the fibroblast. Maintenance of cell by passage culture can be performed in a culture medium added with a substance such as leukemia inhibitory factor (LIF), basic fibroblast growth factor (bFGF)) and the like. The methods for the establishment and maintenance of human and monkey ES cells are described in, for example, U.S. Pat. No. 5,843,780; Thomson J A, et al. (1995), Proc Natl. Acad. Sci. USA. 92:7844-7848; Thomson J A, et al. (1998), Science. 282:1145-1147; H. Suemori et al. (2006), Biochem. Biophys. Res. Commun., 345:926-932; M. Ueno et al. (2006), Proc. Natl. Acad. Sci. USA, 103:9554-9559; H. Suemori et al. (2001), Dev. Dyn., 222:273-279; H. Kawasaki et al. (2002), Proc. Natl. Acad. Sci. USA, 99:1580-1585; Klimanskaya I, et al. (2006), Nature. 444:481-485 and the like.

As a culture medium for generating ES cells, for example, DMEM/F-12 culture medium (or Synthetic medium: mTeSR, Stem Pro and the like) supplemented with 0.1 mM 2-mercaptoethanol, 0.1 mM non-essential amino acid, 2 mM L-glutamic acid, 20% KSR and 4 ng/mL bFGF is used, and human ES cells can be maintained under a wet atmosphere at 37° C., 2% CO₂/98% air (O. Fumitaka et al. (2008), Nat. Biotechnol., 26:215-224). ES cell requires passaging every 3-4 days, and passaging can be performed, for example, using 0.25% trypsin and 0.1 mg/mL collagenase IV in PBS containing 1 mM CaCl₂ and 20% KSR.

ES cell can be generally selected by the Real-Time PCR method by using the expression of a gene marker such as alkaliphosphatase, Oct-3/4, Nanog and the like as an index. Particularly, expression of a gene marker such as OCT-3/4, NANOG, ECAD and the like can be used as an index in selecting human ES cell (E. Kroon et al. (2008), Nat. Biotechnol., 26:443-452).

Human ES cell strain, for example, WA01 (H1) and WA09 (H9), are available from WiCell Research Institute, and KhES-1, KhES-2 and KhES-3 are available from Kyoto University, Institute for Frontier Life and Medical Sciences (Kyoto, Japan).

Spermatogonial stem cell is a pluripotent stem cell derived from the testis, and becomes the origin for spermatozoon formation. Similar to ES cell, this cell can be induced to differentiate into various lines of cells and, for example, has properties permitting creation of a chimeric mouse by transplantation to mouse blastocyst, and the like (M. Kanatsu-Shinohara et al. (2003) Biol. Reprod., 69:612-616; K. Shinohara et al. (2004), Cell, 119:1001-1012). It can self-replicate in a culture medium containing glial cell line-derived neurotrophic factor (GDNF), and produces a spermatogonial cell by repeated passage under culture conditions similar to those for ES cells (Masanori Takebayashi et al. (2008), experiment medicine, vol. 26, No. 5 (special issue), pages 41-46, YODOSHA CO., LTD. (Tokyo, Japan)).

Embryonic germ cell is established from primordial germ cell in the viviparous stage, has pluripotency similar to that of ES cell, and can be established by culturing primordial germ cell in the presence of a substance such as LIF, bFGF, stem cell factor and the like (Y. Matsui et al. (1992), Cell, 70:841-847; J. L. Resnick et al. (1992), Nature, 359:550-551).

Induced pluripotent stem (iPS) cell is an artificial stem cell derived from a somatic cell, which has property almost equivalent to that of ES cell, for example, differentiation pluripotency and proliferative capacity by self-replication, and can be produced by introducing a particular reprogramming factor, in the form of DNA or protein, into somatic cells (K. Takahashi and S. Yamanaka (2006) Cell, 126: 663-676; K.

Takahashi et al. (2007), Cell, 131:861-872; J. Yu et al. (2007), Science, 318:1917-1920; Nakagawa, M. et al. Nat. Biotechnol. 26:101-106 (2008); WO 2007/069666). The reprogramming factor may be constituted of a gene specifically expressed in ES cells, a gene product thereof or non-coding RNA or a gene that plays an important role in maintaining undifferentiation of ES cell, a gene product thereof, or non-coding RNA or a low-molecular weight-compound thereof. Examples of the gene contained in a reprogramming factor include Oct3/4, Sox2, Sox1, Sox3, Sox15, Sox17, Klf4, Klf2, c-Myc, N-Myc, L-Myc, Nanog, Lin28, Fbx15, ERas, ECAT15-2, Tcl1, beta-catenin, Lin28b, Sall1, Sall4, Esrrb, Nr5a2, Tbx3, Glis1 and the like, and these reprogramming factors may be used singly, or in combination. Examples of the combination of reprogramming factors include the combinations described in WO 2007/069666, WO 2008/118820, WO 2009/007852, WO 2009/032194, WO 2009/058413, WO 2009/057831, WO 2009/075119, WO 2009/079007, WO 2009/091659, WO 2009/101084, WO 2009/101407, WO 2009/102983, WO 2009/114949, WO 2009/117439, WO 2009/126250, WO 2009/126251, WO 2009/126655, WO 2009/157593, WO 2010/009015, WO 2010/033906, WO 2010/033920, WO 2010/042800, WO 2010/050626, WO 2010/056831, WO 2010/068955, WO 2010/098419, WO 2010/102267, WO 2010/111409, WO 2010/111422, WO 2010/115050, WO 2010/124290, WO 2010/147395, WO 2010/147612, Huangfu D, et al. (2008), Nat. Biotechnol., 26: 795-797, Shi Y, et al. (2008), Cell Stem Cell, 2: 525-528, Eminli S, et al. (2008), Stem Cells. 26:2467-2474, Huangfu D, et al. (2008), Nat Biotechnol. 26:1269-1275, Shi Y, et al. (2008), Cell Stem Cell, 3, 568-574, Zhao Y, et al. (2008), Cell Stem Cell, 3:475-479, Marson A, (2008), Cell Stem Cell, 3, 132-135, Feng B, et al. (2009), Nat Cell Biol. 11:197-203, R. L. Judson et al., (2009), Nat. Biotech., 27:459-461, Lyssiotis C A, et al. (2009), Proc Natl Acad Sci USA. 106:8912-8917, Kim J B, et al. (2009), Nature. 461:649-643, Ichida J K, et al. (2009), Cell Stem Cell. 5:491-503, Heng J C, et al. (2010), Cell Stem Cell. 6:167-174, Han J, et al. (2010), Nature. 463:1096-1100, Mali P, et al. (2010), Stem Cells. 28:713-720, Maekawa M, et al. (2011), Nature. 474:225-229.

The above-mentioned reprogramming factor also contains a factor used for enhancing the establishment efficiency such as histone deacetylase (HDAC) inhibitor [for example, nucleic acid-based expression inhibitors such as low-molecule inhibitors such as valproic acid (VPA), trichostatin A, sodium butyrate, MC 1293, M344 and the like, siRNA and shRNA for HDAC (e.g., HDAC1 siRNA Smartpool (registered trade mark) (Millipore), HuSH 29 mer shRNA Constructs against HDAC1 (OriGene) etc.) and the like, and the like], MEK inhibitor (e.g., PD184352, PD98059, U0126, SL327 and PD0325901), Glycogen synthase kinase-3 inhibitor (e.g., Bio and CHIR99021), DNA methyltransferase inhibitor (e.g., 5-azacytidine), histone methyltransferase inhibitor (e.g., low-molecule inhibitor such as BIX-01294 and the like, nucleic acid-based expression inhibitors such as siRNA and shRNA for Suv39h1, Suv39h2, SetDBl and G9a and the like, and the like), L-channel calcium agonist (e.g., Bayk8644), butyric acid, TGFβ inhibitor or ALK5 inhibitor (e.g., LY364947, SB431542, 616453 and A-83-01), p53 inhibitor (e.g., siRNA and shRNA for p53), ARID3A inhibitor (e.g., siRNA and shRNA for ARID3A), miRNA such as miR-291-3p, miR-294, miR-295 and mir-302 and the like, Wnt Signaling (e.g., soluble Wnt3a), neuropeptide Y, prostaglandins (e.g., prostaglandin E2 and prostaglandin J2), hTERT, SV40 LT, UTF1, IRX6, GLIS1, PITX2, DMRTB1 and the like. In the present specification, these factors used for improving the establishment efficiency are not specifically distinguished from the reprogramming factor.

The reprogramming factor in a protein form may be introduced into somatic cell by a method, for example, lipofection, fusion with a cellular membrane permeable peptide (e.g., TAT derived from HIV and polyarginine), microinjection and the like.

On the other hand, in the case of a DNA form, for example, it can be introduced into somatic cell by a method using vector (e.g., virus, plasmid, artificial chromosome and the like), lipofection, liposome, microinjection and the like. Examples of the virus vector include retrovirus vector, lentivirus vector (the above: Cell, 126, pp. 663-676, 2006; Cell, 131, pp. 861-872, 2007; Science, 318, pp. 1917-1920, 2007), adenovirus vector (Science, 322, 945-949, 2008), adeno-associated virus vector, Sendai virus vector (WO 2010/008054) and the like. Examples of the artificial chromosome vector include human artificial chromosome (HAC), yeast artificial chromosome (YAC), bacterium artificial chromosome (BAC, PAC) and the like. As a plasmid, a plasmid for mammalian cell can be used (Science, 322:949-953, 2008). The vector can contain regulatory sequences such as promoter, enhancer, ribosome-binding sequence, terminator, polyadenylated site and the like to enable expression of a nuclear reprogramming substance and can contain, where necessary, selection marker sequences such as drug resistance gene (e.g., kanamycin resistance gene, ampicillin resistant gene, puromycin resistance gene and the like), thymidine kinase gene, diphtheriatoxin gene and the like, reporter gene sequences such as green fluorescence protein (GFP), β glucuronidase (GUS), FLAG and the like, and the like. The above-mentioned vector may have a LoxP sequence before and after the vector, to excise a gene encoding a reprogramming factor or both a promoter and a gene encoding a reprogramming factor bonded thereto, after introduction into somatic cell.

In the case of an RNA form, for example, it may be introduced into somatic cell by a method such as lipofection, microinjection and the like, and RNA incorporating 5-methylcytidine and pseudouridine (TriLink Biotechnologies) may be used to suppress decomposition (Warren L, (2010) Cell Stem Cell. 7:618-630).

Examples of the culture medium for induction of iPS cell include DMEM, DMEM/F12 and DME culture media containing 10-15% FBS (these culture media can further contain LIF, penicillin/streptomycin, puromycin, L-glutamine, non-essential amino acids, β-mercaptoethanol and the like as appropriate) or a commercially available culture medium [for example, culture medium for mouse ES cell culture (TX-WES culture medium, Thromb-X), culture medium for primate ES cell culture (culture medium for primate ES/iPS cell, Reprocell Incorporated), serum-free medium (mTeSR, Stemcell Technology) and the like] and the like.

In an example of the culture method, for example, somatic cells and a reprogramming factor are contacted on DMEM or DMEM/F12 culture medium containing 10% FBS at 37° C. in the presence of 5% CO₂ and cultured for about 4-7 days, after which the cells were re-seeded on feeder cells (e.g., mitomycin C treatment STO cell, SNL cell etc.), cultured in a culture medium for bFGF-containing primate ES cell culture from about 10 days after the contact of the somatic cells and the reprogramming factor, and iPS-like colony can be generated in about 30-about 45 days or longer from the contact.

Alternatively, they are cultured on feeder cells (e.g., mitomycin C-treated STO cell, SNL cell etc.) in a 10% FBS-containing DMEM culture medium (which can further contain LIF, penicillin/streptomycin, puromycin, L-glutamine, non-essential amino acids, β-mercaptoethanol and the like as appropriate) at 37° C. in the presence of 5% CO₂ for about 25-about 30 days or longer to generate an ES-like colony. Desirably, a method using a somatic cell itself to be reprogrammed instead of feeder cells (Takahashi K, et al. (2009), PLoS One. 4:e8067 or WO 2010/137746), or extracellular substrate (e.g., Laminin (WO 2009/123349) and Matrigel (BD)) can be recited as an example.

In addition to the above, a culturing method using a medium not containing a serum is also recited as an example (Sun N, et al. (2009), Proc Natl Acad Sci USA. 106:15720-15725). Furthermore, to enhance establishment efficiency, an iPS cell may be established under low oxygen conditions (oxygen concentration of not less than 0.1%, not more than 15%) (Yoshida Y, et al. (2009), Cell Stem Cell. 5:237-241 or WO 2010/013845).

During the above-mentioned culture, from day 2 after the start of culture, the culture medium is exchanged with a fresh culture medium once per day. While the number of somatic cells to be used for nuclear reprogramming is not limited, it is about 5×10³-about 5×10⁶ cells per 100 cm² culture dish.

iPS cell can be selected according to the shape of the colony formed. On the other hand, when a drug resistance gene expressed in association with a gene (e.g., Oct3/4, Nanog) that is expressed when a somatic cell is reprogrammed is introduced as a marker gene, the established iPS cell can be selected by culturing in a culture medium (selective culture medium) containing the corresponding drug. When the marker gene is a fluorescence protein gene, iPS cell can be selected by observation under a fluorescence microscope. When the marker gene is a luminescence enzyme gene, iPS cell can be selected by adding a luminescence substrate, and when it is a chromogenic enzyme gene, iPS cell can be selected by adding a chromogenic substrate.

A clone embryo-derived ES cell (nt ES cell) obtained by nuclear transplantation has almost the same properties as those of fertilized egg-derived ES cell (T. Wakayama et al. (2001), Science, 292:740-743; S. Wakayama et al. (2005), Biol. Reprod., 72:932-936; J. Byrne et al. (2007), Nature, 450:497-502). That is, ES cell established from an inner cell mass of blastocyst derived from cloned embryo obtained by replacing the nucleus of unfertilized egg with the nucleus of somatic cell is nt ES (nuclear transfer ES) cell. For the production of nt ES cell, the nuclear transplantation technique (J. B. Cibelli et al. (1998), Nature Biotechnol., 16:642-646) and the ES cell production technique (mentioned above) are utilized in combination (Kiyoka Wakayama et al. (2008), experiment medicine, vol. 26, No. 5 (special issue), pages 47-52). In nuclear transplantation, the nucleus of somatic cell is injected into an enucleated unfertilized egg of a mammal and cultured for a few hours, whereby reprogramming is performed.

Multilineage-differentiating Stress Enduring cell (Muse cell) is a pluripotent stem cell produced by the method described in WO 2011/007900. To be specific, it is a cell having pluripotency, which is obtained by a trypsin treatment of fibroblast or bone marrow interstitial cell for a long time, preferably 8 hr or 16 hr, followed by suspension culture, and SSEA-3 and CD105 are positive.

### II. Support Composed of Biodegradable Polymer

In the culture substrate of the present invention, the biodegradable polymer constituting the support is not particularly limited as long as it is biocompatible, and maintains the function as a support for a period necessary for the cell population to be transplanted to maintain a functional three-dimensional structure, after transplantation of an agent for cell transplantation containing the culture substrate of the present invention and the cells maintained on the substrate to the living organism to be the target, and is decomposed and disappears. Examples thereof include polyester (e.g., polyglycolic acid (PGA), polylactic acid (PLA), lactic acid-glycolic acid copolymer (PLGA), copolymer of polycaprolactone (PCL) and PGA, block copolymer of PCL and glycoside, lactide, PEG, polydioxane (PDS), polypropylene fumarate (PPF) etc.), polycarbonate (PTMC) and a copolymer thereof (e.g., PTMC, copolymer of trimethylenecarbonate and glycoside, terpolymer of trimethylenecarbonate, glycoside and dioxane etc.), polyanhydride and a copolymer thereof (e.g., melt polycondensation of aliphatic or aromatic dicarboxylic acid, copolymer of polyanhydride and imide etc.), synthetic polymers such as polyorthoester (POE) (e.g., POE I-IV), polyphosphazen (PPZ) and the like, and natural macromolecules such as protein (e.g., gelatin, collagen, laminin, fibroin, keratin etc.), polysaccharide (e.g., agarose, alginic acid, hyaluronic acid, chitin, chitosan etc.). In consideration of the use as an agent for cell transplantation therapy, preferred is one not derived from an animal heterogeneous to the transplantation target, more preferred is a synthetic polymer. Further preferred are polyesters such as PGA, PLA, PLGA and the like, and particularly preferred is PGA.

The above-mentioned synthetic polymers can be produced by a method known per se. For example, in the case of PGA, for example, it can be obtained by ring opening polymerization using glycolide tin octylate and the like as a catalyst. In the case of PLA, it can be obtained by ring opening polymerization using lactide tin octylate and the like as a catalyst. Also, PLGA can be obtained by ring opening copolymerization of lactide and glycolide. In addition, these synthetic polymers are commercially available.

The above-mentioned natural polymers can be each isolated and purified from naturally occurring substances producing them, by a method known per se. When the natural macromolecule is a protein, a recombinant protein is desirably used.

A support made of a biodegradable polymer is preferably flexible and maintains its strength. While the kind of the support is not particularly limited, preferable examples of the support include a fibrous structure (fabric) such as non-woven fabric, knitted fabric, woven fabric and the like, a porous scaffold material, a composite material of a fiber structure and a porous material and the like. More preferred is a fiber structure, and more preferred is a non-woven fabric. The non-woven fabric is a cloth formed without a knitting frame and can be produced by a melt blowing method including blowing a molten macromolecule as fine fibers by air blowing, an electrospinning method or the like. Knitted fabric is a structure in which one fiber is knitted while forming a loop, and a warp knitted mesh which is knitted from a plurality of yarns is also used. A woven fabric is a fabric in which warp yarns and weft yarns are alternately intersected, and gauze and the like are mentioned. As the porous scaffolding material, a porous body obtained by subjecting the above-mentioned biodegradable polymer to a freeze-drying method, an emulsion lyophilization method, a phase separation method, a porogen leaching method, a high pressure gas foaming method, a three-dimensional modeling method, an electrospinning method and the like can be mentioned. As a composite material of a fiber structure and a porous body, one wherein a porous material such as collagen sponge and the like is introduced into the voids of a fiber structure (e.g., knit mesh, braided cord, etc.) of a synthetic polymer such as PLA, PGA and the like can be mentioned.

In one particularly preferable embodiment, the culture substrate of the present invention has PGA non-woven fabric as a support.

When the support is a fiber structure, the fiber constituting the support may have a fiber diameter of 1-100 μm, preferably 2-10 μm, more preferably 2-5 μm. When the support is a fiber structure or a porous body, the pore diameter of the support is not particularly limited as long as it does not adversely affect the culture state of the cells cultured on the culture substrate of the present invention (for example, maintenance, amplification, differentiation, dedifferentiation and the like of cells, preferably maintenance and amplification of stem cells, particularly pluripotent stem cells such as human ES cells, iPS cells and the like, depending on the purpose). For example, when the support is a fiber structure with random fiber direction such as non-woven fabric, the pore diameter of the support may be considerably non-uniform within the range of 5-500 μm, preferably 10-100 μm. On the other hand, when the support is a fiber structure having a constant fiber direction such as a knitted fabric, the pore diameter of the support may be more uniform. The thickness of the support is also not particularly limited as long as it does not adversely affect the culture state of the cells cultured on the culture substrate of the present invention (for example, maintenance, amplification, differentiation, dedifferentiation and the like of cells, preferably maintenance and amplification of stem cells, particularly pluripotent stem cells such as human ES cells, iPS cells and the like, depending on the purpose). For example, the thickness may be 1 μm-3 mm, preferably 10 μm-1 mm, more preferably 50-200 μm.

III. Nanofiber composed of biodegradable polymer

As a biodegradable polymer to be used for a nanofiber of the culture substrate of the present invention, those similar to the biodegradable polymer to be used for the above-mentioned support can be used. Preferred is one not derived from an animal heterogeneous to the transplantation target, more preferred is a synthetic polymer. Gelatin, which is a treated product of a natural macromolecule obtained by chemically treating collagen, is also one preferable embodiment of the present invention.

Gelatin is mainly produced from bovine bone, bovine hide and pig skin as starting materials, but in some cases it may be made from fish skin or squama of salmon and the like as starting materials, and its origin is not particularly limited. Methods for extracting and purifying gelatin from these starting materials are well known. Commercially available gelatin can also be used.

As synthetic polymer, preferred are polyesters such as PGA, PLA, PLGA and the like, and particularly preferred is PGA. These synthetic polymers can be produced as mentioned above, and are also commercially available.

The biodegradable polymer constituting the nanofiber and the biodegradable polymer constituting the support may be the same polymer or different polymers.

While the molecular weight of a biodegradable polymer is not particularly limited, since a nanofiber sometimes cannot be formed by an electrospinning method when the molecular weight is small, for example, it can be appropriately selected within the range of not less than 10 kDa, preferably 20-70 kDa, more preferably 30-40 kDa, in the case of gelatin.

### IV. Production of Nanofiber

A method of producing a nanofiber from these biodegradable polymers is not particularly limited and, for example, electrospinning method, dry spinning method, conjugate molten spinning method, melt-blown method and the like can be recited. An electrospinning method, which is convenient and widely applicable, is preferably used.

When an electrospinning method is used, a biodegradable polymer is first dissolved in a suitable solvent. As the solvent to be used here, any of inorganic solvents and organic solvents can be used as long as it can dissolve a biodegradable polymer. For example, for the production of gelatin nanofiber, acetic acid, formic acid, trifluoroacetic acid and the like are preferably used. In addition, 1,1,1,3,3,3-hexafluoro-2-propanol (HFIP), 2,2,2-trifluoroethanol and the like can be used. For the production of collagen nanofiber, for example, HFIP and the like can be used. On the other hand, for the production of nanofiber composed of a synthetic polymer such as PGA, PLA, PGLA, PCL and the like, methylene chloride, chloroform, HFIP and the like can be used.

While the concentration of the biodegradable polymer solution is not particularly limited, to obtain preferable fiber diameter and uniformity, for example, an acetic acid solution of gelatin is desirably used at a concentration of 5-15 w/v %, preferably 8-12 w/v %, and a HFIP solution of PGA is desirably used at a concentration of 1-10 w/w %, preferably 3-8 w/w %.

The electrospinning method can be performed according to a method known per se. The principle of the electrospinning method is to spray a material by an electric force to form nano-sized fibers. A biological macromolecule solution is filled in a syringe, a nozzle like an injection needle is set on the tip, and a syringe pump is connected to provide a flow rate. A collector for collection of nanofibers is installed at an appropriate distance from the nozzle (The collector may be a flat plate or a wind-up type. A support to be described later may be placed on a flat plate collector to directly form a nanofiber on a support as the culture substrate of the present invention). Then, the positive electrode of the power supply is connected to the nozzle side and the negative electrode is connected to the collector side. When the syringe pump is turned on and the voltage is applied, the biological macromolecule is injected on the collector, and nanofibers are formed. Here, the fiber form and fiber diameter vary depending on the voltage, the distance from the nozzle to the collector, the inner diameter of the nozzle and the like. However, those skilled in the art can appropriately select these and produce uniform nanofibers having a desired fiber diameter. For example, various conditions used in Examples described later can be adopted, or the conditions described in the above-mentioned non-patent documents 4 and 5 can be appropriately used.

A nanofiber produced as mentioned above may have a fiber diameter of 50-5000 nm, preferably 150-1000 nm, more preferably 150-500 nm, further preferably 150-400 nm.

The thickness of the nanofiber is not particularly limited as long as it does not adversely affect the culture state of the cells cultured on the culture substrate of the present invention (for example, maintenance, amplification, differentiation, dedifferentiation and the like of cells, preferably maintenance and amplification of stem cells, particularly pluripotent stem cells such as human ES cells, iPS cells and the like, depending on the purpose). For example, the thickness may be 100-1000 nm, preferably 150-700 nm.

To impart nanofiber with preferable three-dimensional property and facilitate cell dissociation during passage, the resulting nanofiber is preferably subjected to a crosslinking treatment using a suitable crosslinking agent. While the kind of the crosslinking agent is not particularly limited, preferable crosslinking agents include water-soluble carbodiimide (WSC), N-hydroxysuccinimide (NHS) and the like can be mentioned. Two or more kinds of the crosslinking agents may be used in a mixture. A crosslinking treatment can be performed by, for example, dissolving the crosslinking agent in a suitable solvent, and immersing the obtained nanofiber in the crosslinking agent solution. Those of ordinary skill in the art can appropriately determine solution concentration and crosslinking treatment time according to the kind of the crosslinking agent.

When a known peptide conjugate that imparts functionality to the crosslinking agent and the culture substrate, the crosslinking treatment simultaneously affords functional peptide on the nanofiber substrate, which is also useful.

### V. Production of Fiber-On-Fiber

Nanofiber produced as mentioned above is applied on a support, whereby the culture substrate of the present invention (since representative support in the culture substrate is microfiber, those constituted of a support other than fiber structure are also sometimes to be comprehensively referred to as “fiber-on-fiber” in the present specification) can be produced.

While the coating method is not limited as long as nanofibers are applied uniformly on the support, a method of forming nanofibers on a support by a convenient and widely used electrospinning method is preferably used.

The thickness of the fiber-on-fiber is not particularly limited as long as it does not adversely affect the culture state of the cells cultured on the culture substrate of the present invention (for example, maintenance, amplification, differentiation, dedifferentiation and the like of cells, preferably maintenance and amplification of stem cells, particularly pluripotent stem cells such as human ES cells, iPS cells and the like, depending on the purpose). Since the thickness of the nanofiber is sufficiently small relative to the thickness of the support and can be nearly ignored, the thickness of the fiber-on-fiber may be, for example, 1 μm-3 mm, preferably 10 μm-1 mm, more preferably 50-200 μm.

### VI. Culture of Cells by Using Fiber-On-Fiber Substrate

The thus-obtained culture substrate of the present invention (fiber-on-fiber substrate) comprising a nanofiber composed of a biodegradable polymer on a support composed of a biodegradable polymer is used for culturing various cells including stem cells such as pluripotent stem cell and the like (e.g., maintenance and amplification culture, differentiation induction culture, dedifferentiation induction culture and the like). Therefore, the present invention also provides a method of culturing the cells, comprising seeding cells, preferably stem cells, more preferably pluripotent stem cells, on the culture substrate of the present invention and performing stationary culture of the cells.

In the following, the present invention is explained more specifically by taking maintenance and amplification culture method of pluripotent stem cells as an example. Induction of differentiation from pluripotent stem cells or other stem cells into various differentiated cells, dedifferentiation of tissue precursor cells or tissue stem cells, or differentiated cells to a more undifferentiated state, and maintenance and amplification culturing of other stem cells, tissue precursor cells or differentiated cells can also be performed with ease by applying the culture substrate of the present invention instead of the culture substrates used conventionally in a known method.

Firstly, pluripotent stem cells established and subjected to attachment culture on feeder cells and a matrix such as Matrigel, collagen and the like are dissociated by an enzyme treatment, suspended in preferably a medium (those exemplified as the culture medium for pluripotent stem cells in the above-mentioned I. can be similarly used. Preferably, a serum-free medium, more preferably, a medium free of a protein (Xeno-free) derived from an animal heterogeneous to the pluripotent stem cells to be cultured, further preferably, a medium free of protein serum albumin, bFGF and the like is used) added with a ROCK inhibitor (e.g., Y-27632 etc.) to suppress cell death, and seeded on the above-mentioned culture substrate of the present invention, placed in a culture container (e.g., dish, petri dish, tissue culture dish, multidish, microplate, microwell plate, multiplate, multiwell plate, chamber slide, petri dish, tube, tray, culture bag etc.), at a cell density of about 0.5×10⁴-about 10×10⁴ cells/cm², preferably about 2×10⁴-about 6×10⁴ cells/cm². The culture substrate is desirably impregnated, prior to the seeding of the pluripotent stem cells, with a medium having the same composition (ROCK inhibitor is not necessary) as the above-mentioned medium, and pre-incubated under the conditions similar to those of the main culture.

After seeding the pluripotent stem cells, the medium is preferably removed from the culture container and exchanged with a fresh medium (desirably containing ROCK inhibitor), and the cells are cultivated for one day. The culture is performed in, for example, a CO₂ incubator under about 1-about 10%, preferably about 2-about 5%, CO₂ concentration atmosphere, at about 30-about 40° C., preferably about 37° C. The next day, the medium is exchanged with a medium free of a ROCK inhibitor, after which desirably exchanged with a fresh medium every 1-2 days. The culture is performed for 1-7 days, preferably 3-6 days, more preferably 4-5 days.

The present invention also provides a method of culturing cells (e.g., maintenance and amplification method and the like), comprising dissociating cells (e.g., stem cells such as pluripotent stem cell and the like, and the like) from a substrate by using a dissociation solution free of enzyme, re-seeding the cells on the culture substrate of the present invention, and further subjecting the cells to stationary culture. Human pluripotent stem cells are passaged as cell aggregates having a certain size, since they have a problem of easy occurrence of cell death when they are converted to single cells by a conventional passage culture method. When the culture substrate of the present invention is used, cells can be readily dissociated from the substrate by using a dissociation solution free of enzyme, and can be dispersed into single cells by a slight pipetting operation. Using the above-mentioned crosslinked substrate, separation of the substrate from the cell becomes easier since the form of the substrate is retained.

As a dissociation solution free of enzyme, a dissociation solution conventionally used in a method of mechanically dissociating cells can be similarly used and, for example, Hanks' solution, a solution of citric acid and EDTA in combination and the like can be recited.

The points to be noted as regards the present invention is that, when human pluripotent stem cells are dispersed into single cells, the percentage of cell death of the pluripotent stem cells in single cells is markedly suppressed. This makes it possible to prepare cell populations of more uniform human pluripotent stem cells. Therefore, the present invention also provides a method of maintenance and amplification of pluripotent stem cells, which inhibits cell death and enables uniformization of the cells, by dispersing the pluripotent stem cells into single cells by using the culture substrate of the present invention and without performing an enzyme treatment at the time of passage. To disperse the cells dissociated from the substrate into single cells, about 10 times of mild pipetting of the cells in a medium containing a ROCK inhibitor is only required. According to this method, since the death of the cells dispersed into single cells can be remarkably suppressed, addition of a ROCK inhibitor to the medium for about one day is sufficient. Since it is desirable to avoid contact of the ROCK inhibitor with the cells for a long term from the aspect of safety, the effect of the present invention is extremely significant.

Since stem cells, especially human stem cells, are expected to be applicable to transplantation therapy and the like, it is necessary to avoid contamination of viruses and other contaminants harmful to the human body as much as possible to enable safe transplantation. Therefore, particularly in the maintenance and amplification culture of human stem cells, it is desirable to use a serum-free medium, more preferably use of a xeno-free medium not containing components derived from heterologous animals, further preferably use of a protein-free medium. When passage culture is continued using the culture substrate of the present invention, a growth efficiency comparable to that of a serum-containing medium and the like can be obtained even when using any of these media.

Here, Examples of the serum-free medium include mTeSR medium containing transformant animal protein and the like; examples of the xeno-free medium include TeSR2 medium containing human serum albumin; human bFGF and the like, and examples of the protein-free medium include E8 medium and the like.

The pluripotent stem cells (preferably dispersed into single cells) dissociated from the culture substrate of the present invention are seeded on a fresh culture substrate at a cell density of about 0.5×10⁴-about 10×10⁴ cells/cm², preferably about 2×10⁴-about 6×10⁴ cells/cm², at the time of passage culture, similar to the above-mentioned transfer from attachment culture using feeder cells and the like to the culture substrate of the present invention. Similar to the above, the culture substrate is also desirably impregnated, prior to the seeding of the pluripotent stem cells, with a medium having the same composition (ROCK inhibitor is not necessary) as in the main culture, and pre-incubated under the conditions similar to those of the main culture.

After re-seeding the pluripotent stem cells, the medium is preferably removed from the culture container and exchanged with a fresh medium (desirably containing ROCK inhibitor), and the cells are cultivated for one day. The culture is performed in, for example, a CO₂ incubator under about 1-about 10%, preferably about 2-about 5%, CO₂ concentration atmosphere, at about 30-about 40° C., preferably about 37° C. The next day, the medium is exchanged with a medium free of a ROCK inhibitor, after which desirably exchanged with a fresh medium every 1-2 days. The culture is performed for 1-7 days, preferably 3-6 days, more preferably 4-5 days.

By repeating the above operation, it is possible to maintain and amplify pluripotent stem cells with extremely good proliferation efficiency while maintaining pluripotency and normal trait over a long period. As the growth efficiency when human pluripotent stem cells are continuously cultured, the proliferation rate reaches ten times every 5 days. This proliferation rate is strikingly superior to about 5-fold or the like in the paper of dispersion culture of human pluripotent stem cells as previously reported. It is also superior to the conventional laboratory level by complicated manual adhesion culture method (about 4 times every 4 days or about 3 times every 3 days).

In this way, it is possible to stably amplify high quality pluripotent stem cells in large quantities and supply a sufficient amount of pluripotent stem cells as a source of differentiated cells for cell transplantation therapy and drug screening.

### VII. Cryopreservation of Cells by Using Fiber-On-Fiber Substrate

The cells cultured on a fiber-on-fiber substrate can be inserted into a container together with the substrate and cryopreserved. The container may be any as long as it is suitable for freezing, and is not limited by volume, shape (tube, bag, ampoule, vial etc.) and the like. Those of ordinary skill in the art can appropriately select a preferable container. In addition, those of ordinary skill in the art can also change the shape of the substrate after culturing, with tweezers or the like and insert same into a container.

For freezing of cells, those of ordinary skill in the art can add a solution for cell freezing as necessary. The solution may be any as long as it can protect cells under freezing. For example, commercially available products such as mFreSR (VERITAS Corporation), primate ES cell cryopreservation solution (Reprocell Incorporated), CRYO-GOLD Human ESC/iPSC Cryopreservation Medium (System Biosciences), CELL BANKER 3 (JUJI FIELD Inc.) and the like can also be used.

### VIII. Direct Transplantation of Cells Cultured on Fiber-On-Fiber Substrate

Since the culture substrate of the present invention is biocompatible and biodegradable, it can transplant cells cultured on the substrate to the body of animals including human, together with the substrate, without detaching the cells. For example, by using the culture substrate of the present invention, the human pluripotent stem cells maintained and amplified as described above can be induced to differentiate into desired somatic cells on the substrate by exchanging the medium with various differentiation induction media. For example, examples of the differentiation induction method into neural stem cells include the method described in JP-A-2002-291469; examples of the differentiation induction method into pancreatic stem cells include the method described in JP-A-2004-121165; examples of the differentiation induction method into hematopoietic cells include the method described in National Publication of International Patent Application No. 2003-505006, and the like. Besides these, examples of the differentiation induction method by formation of an embryoid body include the method described National Publication of International Patent Application No. 2003-523766 and the like. The somatic cells induced to differentiate in this way can be transplanted together with the substrate without detachment, to the subject, in the same manner as in the conventionally-known transplantation method using a carrier such as a hydrogel and the like. When tumor formation due to the residual undifferentiated cells is concerned, the cell population after differentiation induction is dissociated from the substrate in the same manner as in the ordinary passage, and the undifferentiated cells are removed by flow cytometry or the like by using an undifferentiation marker and/or a differentiation marker, purified to desired somatic cells, reseeded on the culture substrate of the present invention in the same manner as in the ordinary passage, subjected to acclimation culture, and then can also be used for transplantation.

The present invention is explained in more detail in the following by referring to Examples, which are not to be construed as limitative.

### EXAMPLES

### Example 1 Production of Fiber-On-Fiber

### (1) Materials

### Gelatin Solution

gelatin (SIGMA G2625 MW: 30 kDa)
glacial acetic acid (AA; SIGMA P-338826)
anhydrous ethyl acetate (EA; SIGMA P270989)

### Crosslinking Buffer

water-soluble carbodiimide (WSC; DOJINDO Catalog 344-03633)
N-hydroxysuccinimide (NHS; SIGMA Catalog 56480)
0.99.5% ethanol (Wako)
gauze BEMCOT (registered trade mark) S-2 (Asahi Kasei Corporation)
culture cover glass 25 mmφ and 32 mmφ
silicon wafer
vacuum pump
NIPRO blunt needle 23Gx1¼″ non-bevel
high voltage power supply (TECHDEMPAZ Japan)

### (2) Operation Process

### Preparation of 10% w/v Gelatin Solution (AA:EA=3:2) 1 mL

Gelatin (0.1 g) (final concentration 10% w/v), and sterilized distilled water (0.2 mL) were placed in a 2 mL tube. Then, glacial acetic acid (0.42 mL) (final concentration 42% w/v), and anhydrous ethyl acetate (0.31 mL) (final concentration 28% w/v) were added in a draft chamber, and the tube was vortexed and stirred well. When gelatin was sufficiently dissolved, the tube was set on a rotor, and mixed by inverting for one day (room temperature: not less than 20° C.).

### Production of PGA Non-Woven Fabric

According to the method described in Examples 1, 2 of JP-A-2014-083106, and using polyglycolide as a bioabsorbable material, non-woven fabric was produced by a generic compact extruder with a screw diameter 20 mm by a melt-blown method. The inside of the hopper was purged with nitrogen gas, spinning was performed under hot air, and the discharge amount and the speed of the belt conveyor were adjusted to give non-woven fabric. The obtained PGA non-woven fabric had a fiber diameter of 2-5 μm. PGA non-woven fabric having a thickness of 50 μm or 200 μm was subjected to the following production of fiber-on-fiber.

### Application of Gelatin Nanofiber to Support by Electrospinning Method

The gelatin solution prepared as mentioned above was placed in a syringe equipped with a 23G blunt needle (NIPRO) and, after discharging air bubbles, set on a microsyringe pump at a flow rate of 0.2 mL/h. Two pieces of cover glass were placed side by side in the center of silicon wafer (or cotton gauze or PGA non-woven fabric cut into suitable size was placed), and a part of both ends of these supports was fixed with a cellophane tape. The silicon wafer was fixed perpendicularly with vice, and placed at a distance of 10 cm from the needle of the syringe to be set on the microsyringe pump. A positive electrode (red line) was set on the blunt needle, and a negative electrode (green line) was set on the silicon wafer, the microsyringe pump was turned on, and 11 kV voltage was applied to allow the fibers to spout on the support on the silicon wafer. The voltage was stopped, and silicon wafer was rotated 180 degrees to allow for spouting of fiber again for the same time period. After spouting of fibers, the PGA non-woven fabric (fiber-on-fiber the present invention) on the wafer, cotton gauze (control fiber-on-fiber) or glass (control nanofiber) was gently removed and placed in a petri dish. The petri dish was placed in a desiccator, and dried for one day while running the vacuum pump.

### Preparation of 0.2 M WSC/NHS Crosslinking Buffer (40 mL)

WSC (1.52 g), and NHS (0.92 g) were placed in a 50 mL Falcon tube. 99.5% ethanol (30 mL) was added to the tube and vortexed to dissolve the reagent, quantified with 99.5% ethanol to 40 mL, and vortexed again.

### Crosslinking Treatment

Gelatin nanofiber dried in a desiccator (fiber-on-fiber of the present invention, control fiber-on-fiber or control nanofiber) was immersed in a crosslinking buffer in an amount to soak the surface for 4 hr. The nanofiber was taken out, immersed in 99.5% ethanol for 5-10 min and washed (this operation was repeated twice). Then, the nanofiber was air-dried on a petri dish with KimWipes, placed in a desiccator and dried for one day.

### (3) Results

### Structure of Fiber-On-Fiber

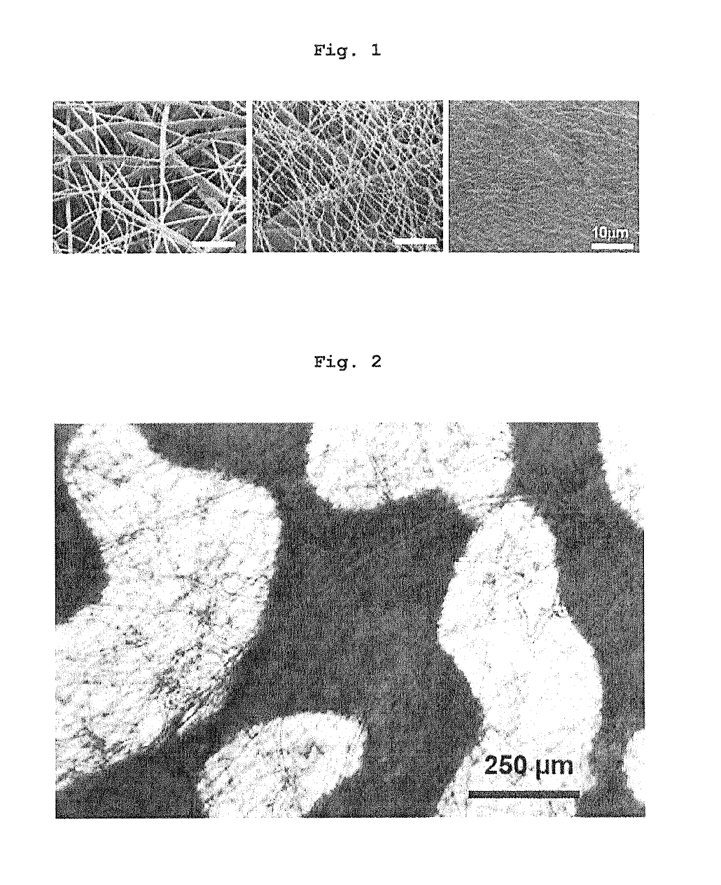

A scanning electron micrograph of the fiber-on-fiber of the present invention having PGA non-woven fabric obtained by the aforementioned method as a support is shown in FIG. 1. It was clarified that the gelatin nanofiber formed a mesh shape between fibers of the PGA non-woven fabric. The gelatin nanofiber had a diameter of 300±100 nm.

### Example 2 Passage Method of Human Pluripotent Stem Cells on Fiber-On-Fiber

### (1) Materials

mTeSR1 STEM cell VERITAS Corporation ST-05850
Y-27632 Wako 257-00511 (1 mg) 253-00513 (5 mg)
Cell Dissociation Buffer enzyme-free, Hanks′-based GIBCO 13150-016
TrypLE Express GIBCO 12605-010
human embryonic stem cells: H9, H1
human induced pluripotent stem cell: 253G1

### (2) Operation Process

### Pre-Treatment of Nanofiber

Various nanofibers prepared in Example 1 were set on 35 mm dish (6-well plate), washed 3 times with 99.5% ethanol (1 mL), 3 times for sterilization treatment. In the third time, ethanol was carefully aspirated, and dried in clean bench. Various nanofibers were immersed in a medium, and incubated at 37° C. mTeSR1 (2 mL) was placed in a 35 mm dish.

Transfer of Human Pluripotent Stem Cells from MEF Feeder onto Nanofiber

To human pluripotent stem cell colony (60 mm dish) on MEF feeder was added enzyme dissociation solution TrypLE Express (2 mL), and the mixture was incubated as it was. About 2 min later, the dish was shaken and delamination of MEF and round colony were confirmed under a microscope, after which the enzyme dissociation solution was removed by aspiration (rinsed with mTeSR1 (1-2 mL) as necessary). The cells were recovered with 10 μM Y-27632-containing mTeSR1 (mTeSR1 (+Y-27632)) (4 mL), and pipetted about 10 times to give single cells. The cell number was counted, the cells were centrifuged at 1000 rpm for 3 min, the supernatant was removed by aspiration, and the cells were resuspended in mTeSR1 (+Y-27632) at a necessary cell concentration. The medium on the pre-treated control nanofiber was removed by aspiration, and 1-1.5 mL (cell density was 2×10⁵-3×10⁵ cells/sample) was seeded on the nanofiber. The next day, the medium was exchanged with mTeSR1 (+Y-27632) (2 mL), cultured in mTeSR1 free of Y-27632 from day 2, and the medium was exchanged every day.

Passage of Colony from Nanofiber to Nanofiber

The cells were rinsed twice with PBS, an enzyme-free cell dissociation solution Cell Dissociation Buffer (1 mL) was added, and the mixture was incubated at 37° C. for 5 min, and the dissociation solution was removed by aspiration (when TrypLE Express was used, 1 mL was added, immediately removed by aspiration and incubated for about 2 min). The cells were recovered with mTeSR1 (+Y-27632) (2 mL) (1 mL×2 times) and pipetted about 10 times to give single cells. The operation thereafter was similar to that in transfer from MEF feeder.

Passage of Colony from Nanofiber to Fiber-On-Fiber

The cells passaged not less than 20 times on the control nanofiber were rinsed twice with D-PBS. Cell Dissociation Buffer (1 mL) was added, and the mixture was incubated at 37° C. for 5 min, and removed by aspiration. The cells were recovered with mTeSR1 (+Y-27632) (2 mL) (1 mL×2 times), and pipetted about 10 times to give single cells. The cell number was counted, the cells were centrifuged at 1000 rpm for 3 min, the supernatant was removed by aspiration, and the cells were resuspended in mTeSR1 (+Y-27632) at a necessary cell concentration. The cell suspension was seeded on fiber-on-fiber (2 cm×2.5 cm) at 2×10⁵ cells/sample. The cells were cultured in mTeSR1 free of Y-27632 from day 2, and the medium was exchanged every day. After 3 days, the cells were used for transplantation.

### (3) Results

### Culture of Human Pluripotent Stem Cells on Fiber-On-Fiber

The fiber-on-fiber of the present invention was immersed in a culture medium, and human pluripotent stem cells (H1 human ES cells) were cultured thereon. Formation of colony by human pluripotent stem cells was confirmed. The results of alkaline phosphatase (pluripotent stem cell marker) staining of the cells are shown in FIG. 2. Colonies stained red were observed, and it was confirmed that human pluripotent stem cells strongly express alkaline phosphatase even after culturing. Moreover, the stained cells were uniformly dispersed on the fibers.

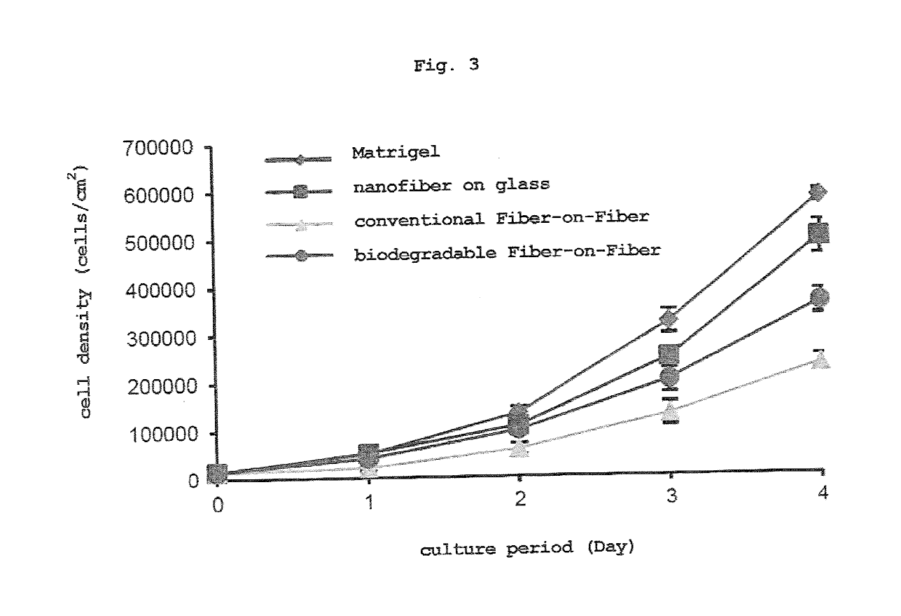

Human ES cells (H1) were cultured for 4 days on the fiber-on-fiber of the present invention, fiber-on-fiber having cotton gauze as a support, and gelatin nanofiber formed on glass, and the cell density was measured every other day. As a result, the cell proliferation efficiency was remarkably improved by using PGA non-woven fabric as a support as compared to cotton gauze as a support, and a proliferation rate close to that of Matrigel and nanofiber on glass was obtained (FIG. 3).

### Quantitative Analysis of Expression Level of Pluripotent Stem Cell Marker by Flow Cytometry

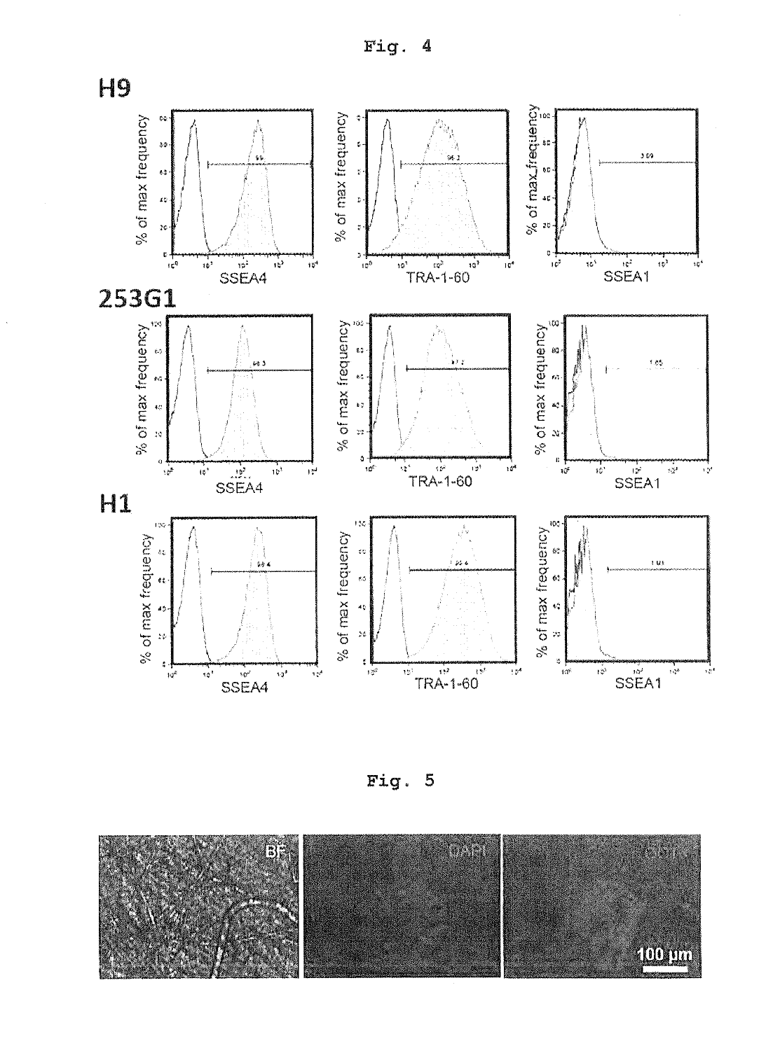

Human ES cells (H1, H9) and human iPS cells (253G1) were cultured on the fiber-on-fiber of the present invention and the expression of pluripotent stem cell markers (SSEA4, TRA-1-60) and differentiation marker (SSEA1) in the cells was analyzed by flow cytometry (FIG. 4). It could be confirmed that, in any pluripotent stem cells, not less than 96% of the cells strongly expressed undifferentiated marker. It could also be confirmed that the cell population was uniform.

### Confirmation of Pluripotent Stem Cell Marker Expression by Immunocyte Staining Method

Human ES cells (H1) were cultured on the fiber-on-fiber of the present invention and the expression of undifferentiated marker (OCT4) and differentiation marker (SSEA1) in the cells was analyzed by immunocyte staining. The results are shown in FIG. 5. It was confirmed that the cells strongly expressed the undifferentiated marker.

### Example 3 Transplantation of Human Pluripotent Stem Cells Cultured on Fiber-On-Fiber to Mouse

### (1) Materials

isoflurane: ABBOTT JAPAN CO., LTD.
immunodeficient mouse: CLEA Japan, Inc.
Natsume atraumatic needle (sterilized): Natsume Seisakusho Co., Ltd.

### (2) Operation Process

A fiber-on-fiber having PGA non-woven fabric as a support carrying cultured human pluripotent stem cells obtained in Example 2 was cut into 2×2.5 cm square.

Immunodeficient mouse (SCID C.B-17/icr-scid/scid Jcl mouse, 8-week-old, female) was placed under systemic anesthesia by inhalation anesthesia with isoflurane. When the mouse was completely at rest, the skin of dorsal flank was incised by about 1 cm. Using tweezers, the above-mentioned fiber-on-fiber was folded about 3 times, inserted into the incised position, and the transplantation site was sutured using a Natsume atraumatic needle. When the teratoma grew to 2 cm in size (1-2 months later), it was removed from the mouse, immobilized by a conventional method. Sections were produced and subjected to Hematoxylin-Eosin staining.

### (3) Results

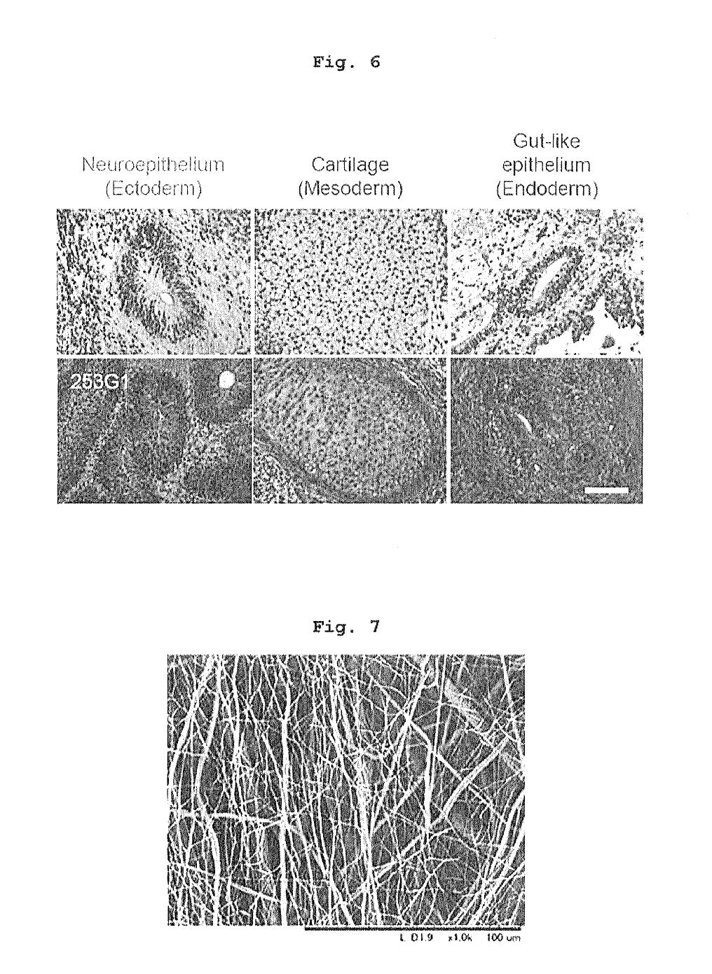

A fiber-on-fiber of the present invention carrying cultured human ES cells (H1) or human iPS cells (253G1) obtained in Example 2 was transplanted to immunodeficient mouse, and the formation of teratoma was examined. In both cells, teratoma containing three germ layers was formed, and it could be confirmed that the fiber-on-fiber of the present invention does not inhibit differentiation of human pluripotent stem cells (FIG. 6). In addition, necrosis was not developed during transplantation, and a post-transplantation inflammation reaction was not observed. Furthermore, the fiber-on-fiber of the present invention completely disappeared in teratoma.

### Example 4 Production of Fiber-On-Fiber Constituted of PGA Alone

### (1) Materials

PGA solution
   PGA
   1,1,1,3,3,3-hexafluoro-2-propanol (HFIP; Wako 085-04235)

### (2) Operation Process

PGA non-woven fabric was produced in the same manner as in Example 1. Preparation of PGA solution and application of PGA nanofiber to a support were performed by the following process.

### 5. Preparation of 7% w/w PGA Solution

PGA (2.85 g) and HFIP (47.15 g) (final concentration 5.7% w/w) were placed in a 50 mL bottle, and the mixture was stood at 50° C. overnight to dissolve PGA.

### Application of PGA Nanofiber to Support by Electrospinning Method

PGA solution prepared as mentioned above was placed in a syringe equipped with a 28G metal needle, and set on an electrospinning apparatus. A metal table was configured at a distance of about 10 cm from the metal needle, and PGA non-woven fabric was fixed with cellophane tape. Air pressure was applied inside the syringe to allow for discharge of the PGA solution, on which a voltage was applied, to give a PGA fiber.

### (3) Results

### Structure of Fiber-On-Fiber Constituted of PGA Alone

A scanning electron micrograph of the fiber-on-fiber constituted of PGA alone of the present invention having PGA non-woven fabric as a support obtained by the aforementioned method is shown in FIG. 7. It was clarified that the PGA nanofiber was a mesh shape formed on the PGA non-woven fabric. The PGA nanofiber had a diameter of 400±100 nm.

### Example 5 Culture of Human Pluripotent Stem Cell on Fiber-On-Fiber Constituted of PGA Alone

(1) Materials mTeSR1 STEM cell VERITAS Corporation ST-05850
Y-27632 Wako 257-00511 (1 mg) 253-00513 (5 mg)
Cell Dissociation Buffer enzyme-free, Hanks'-based GIBCO 13150-016
TrypLE Express GIBCO 12605-010
human induced pluripotent stem cell: 253G1

### (2) Operation Process

By a process similar to that in Example 2, human iPS cells (253G1) were cultured on the fiber-on-fiber constituted of PGA alone produced in Example 4.

### (3) Results

### Alkaline Phosphatase Staining

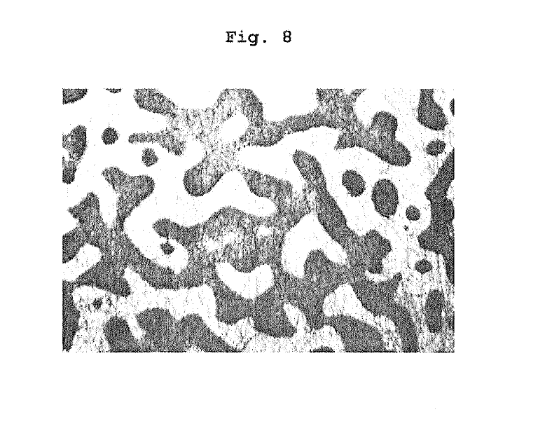

The results of alkaline phosphatase (pluripotent stem cell marker) staining of the human iPS cells (253G1) after culturing are shown in FIG. 8. Stained colonies were observed, and it was confirmed that human iPS cells (253G1) strongly express alkaline phosphatase even after culturing. Moreover, the stained cells were uniformly dispersed on the fibers.

### Quantitative Analysis of Expression Level of Undifferentiated Marker by Flow Cytometry

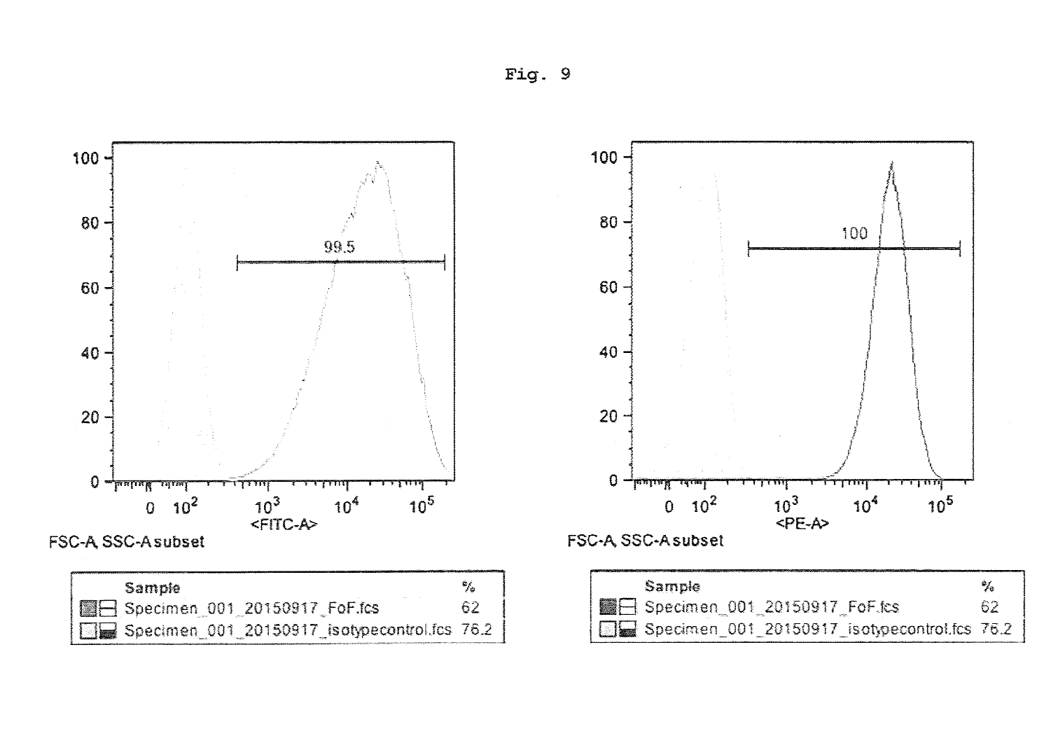

The expression of an undifferentiation marker (TRA-1-60, SSEA4) in human iPS cells (253G1) after culture was analyzed by flow cytometry (FIG. 9). It could be confirmed that, in any pluripotent stem cells, not less than 99.5% of the cells strongly expressed both markers of TRA-1-60 (FIG. 9, left) and SSEA4 (FIG. 9, right).

### Example 6 Confirmation of Substance Diffusion Behavior Via Fiber-On-Fiber Constituted of PGA Alone

### (1) Materials

red food coloring, YOUKI MC food color box (YOUKI FOOD CO., LTD., 52100071077)
phosphate buffered saline D-PBS (Invitrogen, 14287-080)
fiber-on-fiber constituted of PGA alone

### (2) Operation Process

One sheet of the fiber-on-fiber constituted of PGA alone produced in Example 4 was sandwiched between a 1.5 mL tube containing a red food coloring solution and a 1.5 mL tube containing phosphate buffered saline, and the tubes were connected and stood for 3 hr.

### (3) Results

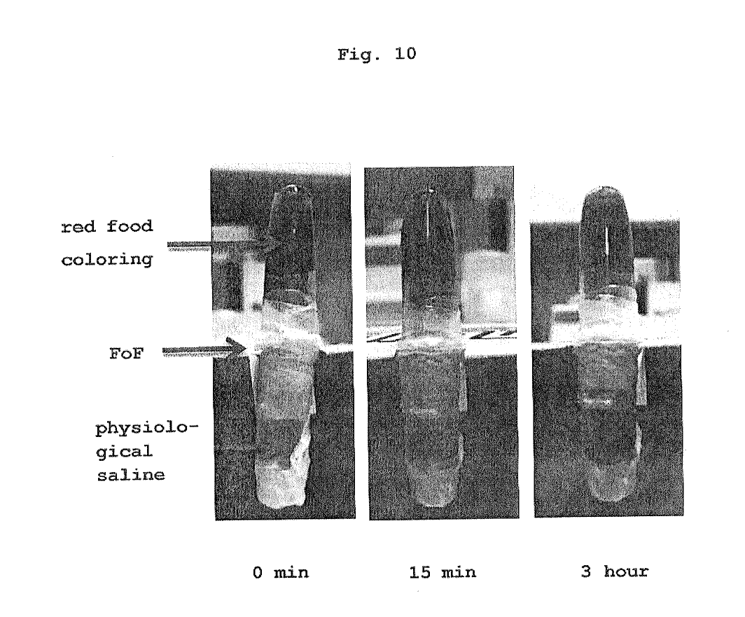

It was confirmed that the red food coloring reached the tube containing phosphate buffered saline 15 min later through the fiber-on-fiber constituted of PGA alone (FoF) (FIG. 10). Therefrom it was suggested that, unlike the use of general culture dish, the cells can obtain the necessary components from any angle of 360 degrees and can release unnecessary substance. Therefore, diffusion of growth factor, supplement, gas molecule and the like contained in the culture medium through the fiber-on-fiber is considered to be similarly possible.

### INDUSTRIAL APPLICABILITY

It is extremely simple and effective, as compared to conventional or known methods, for the design and integration of a mass culture apparatus, particularly an automated culture apparatus, indispensable for the practicalization of human pluripotent stem cells for medicine and drug discovery, and can be utilized for the development of such culture apparatuses. In consideration of the application and development of human pluripotent stem cells for cell transplantation therapy, regenerative medicine and the like, nanofiber that enables three-dimensional culture plays a highly important role.

While the present invention has been described with emphasis on preferred embodiments, it is obvious to those skilled in the art that the preferred embodiments can be modified. The present invention intends that the present invention can be embodied by methods other than those described in detail in the present specification. Accordingly, the present invention encompasses all modifications encompassed in the gist and scope of the appended “CLAIMS.”

The contents disclosed in any publication cited herein, including patents and patent applications, are hereby incorporated in their entireties by reference, to the extent that they have been disclosed herein.

This application is based on a patent application No. 2014-223702 filed in Japan (filing date: Oct. 31, 2014), the contents of which are incorporated in full herein.

1. A cell culture substrate comprising a nanofiber composed of a biodegradable polymer on a support composed of a biodegradable polymer.

2. The substrate according to claim 1, wherein the biodegradable polymer constituting the support is a synthetic polymer.

3. The substrate according to claim 2, wherein the synthetic polymer is polyglycolic acid (PGA).

4. The substrate according to claim 1, wherein the support is a non-woven fabric.

5. The substrate according to claim 1, wherein the biodegradable polymer constituting the nanofiber is gelatin or a synthetic polymer.

6. The substrate according to claim 5, wherein the synthetic polymer is PGA.

7. The substrate according to claim 1, wherein the cell is a pluripotent stem cell.

8. A method of culturing cells comprising seeding cells on the substrate according to claim 1, and subjecting the cells to stationary culture.

9. The method according to claim 8, wherein the cell is a pluripotent stem cell.

10. An agent for a cell transplantation therapy, comprising the substrate according to claim 1, and cells cultured on the substrate.

11. The agent according to claim 10, wherein the cell was induced to differentiate from a pluripotent stem cell.
